(19) United States
(12) Patent Application Publication
(10) Pub. No.: US 2004/0018589 A1
(43) Pub. Date: Jan. 29, 2004

(54) Method for producing biologically active botulinum neurotoxins through recombinant DNA technique

(75) Inventor: Jun Zhong, New York, NY (US)

Correspondence Address: Jun Zhong, 230 Riverside Dr. #12M, New York, NY, 10025, US

(73) Assignee: Jun Zhong, New York, NY

(21) Appl. No.: 10/205,516

(22) Filed: Jul. 25, 2002

(51) Int. Cl. C12P 21/02; C12N 1/21

(52) U.S. Cl. 435/69.1; 435/252.3

(57) ABSTRACT

An artificial sequence that corresponds to the cleavage site for a sequence-specific protease is inserted into the botulinum toxin genes to ensure efficient cleavage of the inactive holotoxins and the production of the active light-chain and heavy-chain duplex toxins.

### CROSS-REFERENCE TO RELATED APPLICATIONS

Reference Cited:

U.S. Patent Documents:

U.S. Pat. No. 5,512,547 April 1996 Johnson and Goodnough 514/21

### OTHER REFERENCES

Montecucco, C. et al., “Botulinum neurotoxins: mechanism of action and therapeutic applications”, Molecular Medicine Today, October 1996, pp.418-424.

Kiyatkin, N. et al., “induction of an immune response by oral administration of recombinant botulinum toxin”, Infection and Immunity, Vol. 65, November 1997, pp.4586-4591.

Binz, T. et al., “The complete sequence of Botulinum Neurotoxin Type A and Comparison with other Clostridial Neurotoxins”, J. Biological Chemistry, Vol. 265, Jun. 5, 1990, pp.9153-9158.

### STATEMENT REGARDING FEDERALLY SPONSORED RESEARCH OR DEVELOPMENT

This invention is not made under any federally sponsored research and development.

### REFERENCE TO A SEQUENCE LISTING, A TABLE, OR A COMPUTER PROGRAM LISTING COMPACT DISC APPENDIX

Not Applicable

### BACKGROUND OF THE INVENTION

The invention relates to the process of producing botulinum toxins in a man-made system. It describes a crucial step that renders the biologically active toxins.

Botulism is one of the most serious forms of food poisoning associated with the ingestion of inappropriately preserved meat products. The symptoms of botulism are caused by the neurotoxins secreted by the Gram-positive bacterium Clostridium botulinum. Seven different botulinum neurotoxins have been identified so far (A-G). Although they differ antigenically, these neurotoxins are closely related and similar to each other in term of their structures and toxic activity.

The botulinum toxins are produced in Clostridium botulinum under anaerobic conditions. They are first synthesized as inactive single-chain peptides (referred to as holotoxins) with molecular masses of ˜150 kDa. The inactive holotoxins are subsequently activated by proteolytic cleavage that produces the 50-kDa light chains and the 100-kDa heavy chains (reviewed by Montecucco et al., 1996). The light chains remain bound to the heavy chains via a disulfide bond after the cleavage. Upon penetrating the intestinal epithelial layer and entering the bloodstream, the heavy chains bind specifically to the receptors on the nerve terminals that project to the muscle, and lead to the internalization of the toxins. The light chains of the botulinum toxins are target-specific protease that, once inside the cell, dissociate from the heavy chains and cleave various components of the SNARE complex required for neurotransmitter release. Thus the botulinum toxins paralyze their victims by blocking the nerve cells that control the muscle contraction.

The ability of the botulinum toxins to block muscle contraction is utilized by doctors to treat spasticity, the overactivity in muscles that could cause pain and deformity. More recently, dermatologists began to use the botulinum toxins to remove wrinkles on the forehead for cosmetic purposes. The type A botulinum toxin is most commonly used for these purposes. It is currently produced and purified from type A Clostridium botulinum as protein complexes that contain the neurotoxin and various nontoxic accessory proteins. Although these accessory proteins may contribute to the stability of the toxin, the large sizes of these complexes are also more likely to activate the immune system. The production of neutralizing antibodies in the patients would render the same toxin complex useless for repeated injections. Johnson and Goodnough (1996) described a pharmaceutical composition consisting of pure type A botulinum neurotoxin and stabilizing agents that resulted in higher specific toxicity and lower antigenity than the toxin complexes.

In addition to being more antigenic and less uniform, the toxin complexes are also difficult to produce and purify. It would be advantageous if one can produce high-purity botulinum neurotoxins suitable for medical use through recombinant DNA technique. Recombinant DNA technique allows high level expression of the target proteins in a controlled manner, through manipulating the DNA fragments containing the genetic information. It also allows adding enhanced features to the toxins through genetic engineering, such as higher stability and longer half-life inside the cell. Kiyatkin et al (1997) have expressed and purified an inactivated form of type C botulinum toxin in an E. coli expression system. However, E. coli did not provide efficient cleavage of the holotoxin. Therefore, to produce biologically active botulinum neurotoxins through the recombinant DNA technique, one must overcome this obstacle and provide a reliable method to transform the non-active holotoxins into the active light-chain and heavy-chain duplexes.

### BRIEF SUMMARY OF THE INVENTION

To ensure efficient proteolytic cleavage of the recombinant botulinum holotoxins, I invented this method of introducing a specific proteolytic site between the coding sequences for the light chains and the heavy chains of the botulinum toxins. The resulting DNA constructs are used to produce the full-length botulinum holotoxins in an expression system. Treatment of the recombinant holotoxins with the protease that recognizes and cuts at the engineered proteolytic site will ensure efficient cleavage of the holotoxins, and render them their biological activities.

The invention makes it possible to produce high-purity and highly uniform botulinum neurotoxins through recombinant DNA techniques, which may result in higher specific-activity and less antigenic products. The invention also makes it easier to produce botulinum toxins with enhanced features such as higher stability and longer half-life inside the cell, which will reduce the frequency and the amount of toxin needed for injections.

### BRIEF DESCRIPTION OF THE DRAWINGS

Not Applicable

### DETAILED DESCRIPTION OF THE INVENTION

For the purpose of demonstration, I use botulinum toxin A as an example, but the same principle should also apply to botulinum toxin B, C1, D, E, F, and G, since they are similar to each other in term of structure and functionality (see Montecucco et al., 1996). For the same reason, I use the Factor Xa site as an example, although any other proteolytic site should work as well. I choose to demonstrate how a specific site could be inserted into the botulinum toxin type A gene through recombinant DNA techniques, although the similar results could also be achieved through other methods such as mutagensis and selection.

The DNA sequence coding for type A botulinum toxin was published in 1990 by Binz et al. The holotoxin is cleaved between Lys⁴⁴⁸ and Ala⁴⁴⁹ in Clostridium botulinum, and the resulting light chain and heavy chain are linked to each other via a disulfide bond formed between Cys⁴³⁰ and Cys⁴⁵⁴. I choose to insert a Factor Xa site between the Lys⁴⁴⁸ and Ala⁴⁴⁹, although other locations between Cys⁴³⁰ and Cys⁴⁵⁴ may also work. The Factor Xa protease recognizes the amino acid sequence of “Ile-Glu-Gly-Arg”, and cuts after the Arginine. According to the rules of universal codon usage, a DNA sequence of “ATA GAA GGG AGA” would encode for amino acids “Ile-Glu-Gly-Arg”. The sense and anti-sense DNA oligonucleotides (“5′ ATA GAA GGG AGA 3′ ” and “5′ TCT CCC TTC TAT 3′ ”, respectively) arc synthesized in vitro, and allowed to anneal to each other to form a double-stranded DNA fragment. This double-stranded DNA fragment should then be ligated to the DNA fragments containing codon 1-448 and codon 449-1296 of the botulinum toxin type A gene. Similarly, a DNA fragment encoding for six histidines (“5′ CAC CAT CAC CAT CAC CAT 3′”) should be ligated to the end of the recombinant toxin, before the stop codon, for the purpose of purification. The DNA fragment encoding the recombinant toxin should then be cloned into a bacterial expression vector and transfected into an appropriate host strain of E. coli.

Many commercially available expression systems can be used to produce the recombinant holotoxin, and the protocols recommended by the manufacturers should be followed. One of such expression systems is the one used by Kiyatkin et al (1997). Briefly, after transfecting the recombinant DNA construct into the E. coli host, the bacteria should be grown in LB medium until they reach exponential-growth phase, with an absorbance at 600 nm around 0.6. Isopropyl-beta-D-thiogalactopyranoside (IPTG) is then added to the medium to induce the expression of the holotoxin. E. coli cells are then harvested from the culture and lysed on ice by sonication. The bacterial lysates should then be cleared by centrifugation and passed through a nickel column. The 6-histidine tag will allow the recombinant holotoxin to bind to the nickel column and be separated from other bacterial proteins. The purified holotoxin should then be incubated with the Factor Xa protease (Pierce, Rockford, Ill.). This will allow sufficient cleavage between the light and the heavy chains. The resulting toxin should be analyzed for its concentration, purity, and specific toxic activity.

Although the E. coli expression systems may be ideal for this purpose, other expression systems may also be used. In addition, the recombinant toxin can be expressed in an E. coli strain that produces the Factor Xa protease, so that the toxin will be cleaved inside the bacterial cell. In that case, the final step of in vitro protease treatment will not be needed.

The complete sequences for all seven botulinum neurotoxins are available online from the National Center for Biotechnology Information (http://www.ncbi.nlm.nih.gov/). For botulinum toxin B, the disulfide bond is formed between Cys⁴³⁷ and Cys⁴⁴⁶, and the natural cleavage site is between Lys⁴⁴¹ and Ala⁴⁴². For botulinum toxin C1, the disulfide bond is formed between Cys⁴³⁷ and Cys⁴⁵³, and the natural cleavage site is between Lys⁴⁴⁹ and Thr⁴⁵⁰. For botulinum toxin D, the disulfide bond is formed between Cys⁴³⁷ and Cys⁴⁵⁰, and the natural cleavage site is between Lys⁴⁴² and Asp⁴⁴³. For botulinum toxin E, the disulfide bond is formed between Cys⁴¹² and Cys⁴²⁶, and the natural cleavage site is between Arg⁴²² and Lys⁴²³. For botulinum toxin F, the disulfide bond is formed between Cys⁴²⁹ and Cys⁴⁴⁵, and the natural cleavage site is between Lys⁴³⁶ and Gly⁴³⁷. For botulinum toxin G, the disulfide bond is formed between Cys⁴³⁶ and Cys⁴⁵⁰, and the natural cleavage site is between Lys⁴⁴² and Asp⁴⁴³. I would first choose to insert the proteolytic sequence at the natural cleavage site, although any other location between the two Cysteines that form the disulfide bond may work as well.

What is claimed is:

1. A process that includes the introduction of a sequence-specific proteolytic site into the natural or genetically modified type A botulinum toxin, so that after proteolytic cleavage, results in the production of a biologically active light-chain and heavy-chain duplex neurotoxin.

2. The process of claim 1 wherein the type A botulinum toxin is replaced by type B botulinum toxin.

3. The process of claim 1 wherein the type A botulinum toxin is replaced by type C1 botulinum toxin.

4. The process of claim 1 wherein the type A botulinum toxin is replaced by type D botulinum toxin.

5. The process of claim 1 wherein the type A botulinum toxin is replaced by type E botulinum toxin.

6. The process of claim 1 wherein the type A botulinum toxin is replaced by type F botulinum toxin.

7. The process of claim 1 wherein the type A botulinum toxin is replaced by type G botulinum toxin.

8. The process of claim 1 wherein the light chain of the natural or genetically modified botulinum toxin type A, or B, or C1, or D, or E, or F, or G is linked to the heavy chain of another natural or genetically modified botulinum toxin by a sequence that contains a sequence-specific proteolytic site.
